(19) United States
(12) Patent Application Publication
(10) Pub. No.: US 2015/0283051 A1
(43) Pub. Date: Oct. 8, 2015

(54) DERIVATIVE OF NOVEL POLYHYDROXY AROMATIC COMPOUND AND USE THEREOF

(71) Applicant: RNS CO.,LTD., Daejeon (KR)

(72) Inventor: Cheong Taek Kim, Daejeon (KR)

(21) Appl. No.: 14/441,557

(22) PCT Filed: Nov. 15, 2013
(86) PCT No.: PCT/KR2013/010412

§ 371 Date: May 8, 2015

(30) Foreign Application Priority Data
Nov. 15, 2012 (KR) 10-2012-0129687

(51) Int. Cl. A61K 8/37; C07C 69/533; A61Q 19/02; C07D 311/22; A61K 8/49; C07C 69/28; C07D 213/69

(52) U.S. Cl. CPC A61K 8/37; C07C 69/28; C07C 69/533; C07D 213/69; C07D 311/22; A61K 8/4926; A61K 8/498; A61Q 19/02

(57) ABSTRACT

Provided is a skin whitening composition with excellent whitening effects, specifically, a derivative or a polyhydroxy cyclic compound represented by Formula I or a pharmacologically acceptable salt thereof, comprising;

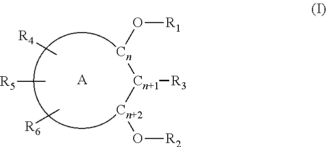

wherein

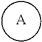

is derived from an aromatic cyclic compound, Cₙ, Cₙ₊₁ and Cₙ₊₂ are three neighboring carbon atoms present in the aromatic cyclic compound, wherein n is a positive integer, R₁ and R₂ are each a saturated or unsaturated straight or branched alkyl or acyl group having 3 to 12 carbon atoms, and R₃, R₄, R₅ and R₆ are each independently at least one substituent selected from hydrogen, alkyl, alkoxy, acyloxy, acyloxymethyl, oxo, hydroxy, vinyl, nitrile, carboxaldehyde, carbonitrile and aldehyde.

### TECHNICAL FIELD

The present invention relates to a novel derivative of a cyclic compound, and more particularly, to a topical skin treatment agent for external application, in particular, a topical treatment agent composition for external application to the skin with excellent whitening effects.

### BACKGROUND ART

Melanogenesis is a result of a process wherein generation of melanin is increased by a protective mechanism in melanocytes resulting from various forms of stimulation such as UV light, which in turn, causes a large amount of the melanin generated thereby to be transited to tokeratinocytes, and then accumulated in an epidermal layer of skin. Although melanin functions to protect the skin, hyper-pigmentation of the skin may cause liver spots, freckles, melanogenesis after dermal inflammation, senile pigment spots, or the like, hence incurring not only cosmetic inconveniences but also negative mental effects and, in turn, discomfort in the social life of a person suffering from the above problem. It is known that the process of melanin generation is generally performed by an enzyme called tyrosinase acting on tyrosine, which is one type of amino acid, to convert it into dopa and then dopaquinone, which in turn, undergoes non-enzymatic oxidation. It is considered that such a generated melanin may be abnormally deposited in the skin so as to cause liver spots, age spots, or the like. In order to alleviate, prevent and/or treat pigmentation, freckles and/or spots, etc., some substances for inhibiting the generation of melanin, for example, hydroquinone, arbutin, vitamin C and derivatives thereof have been developed and whitening cosmetics containing the same are also known to the public. Korean Patent Laid-Open Publication No. 2005-0509848 discloses a whitening cosmetic containing keratone derivatives separated from Anthrisci radix, and Korean Patent Laid-Open Publication No. 2005-0479741 discloses a whitening cosmetic containing glucose acylated derivatives. Among these, although hydroquinone is recognized to have some beneficial effects, its use is often limited due to sensitizing properties. Also, ascorbic acid is apt to be oxidized and a cosmetic combined with the same may cause problems such as discoloration or a change in scent. And, in general, since plant extract-derived substances involve considerable differences in efficacies depending on the origins of the plants, it is difficult to maintain uniformity of products. Further, glucose acylated derivatives have a drawback of very low synthesis efficiency.

Therefore, as a result of continuous studies to develop a material capable of inhibiting melanin generation, the present inventors have synthesized novel cyclic compound derivatives and found that these derivatives exhibit excellent whitening effects, thereby completing the present invention.

### SUMMARY OF THE INVENTION

### Problems to be Solved by the Invention

Accordingly, it is an object of the present invention to provide a skin whitening composition that is easily synthesized and exhibits excellent effects of inhibiting melanin generation with no adverse effect on the skin, so as to provide superior skin pigmentation inhibitory effects.

### Means for Solving the Problems

In order to accomplish the above objects, according to one aspect of the present invention, there is provided a derivative of a polyhydroxy cyclic compound represented by Formula I below or a pharmacologically acceptable salt thereof, including:

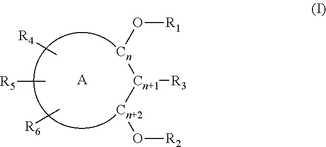

wherein

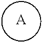

is derived from an aromatic cyclic compound,

Cₙ, Cₙ₊₁ and Cₙ₊₂ are three neighboring carbon atoms present in the aromatic cyclic compound, wherein n is a positive integer,

R₁ and R₂ are each a saturated or unsaturated straight or branched alkyl or acyl group having 3 to 12 carbon atoms, and

R₃, R₄, R₅ and R₆ are each independently at least one substituent selected from a group consisting of hydrogen, alkyl, alkoxy, acyloxy, acyloxymethyl, oxo, hydroxy, vinyl, nitrile, carboxaldehyde, carbonitrile and aldehyde.

### Effects of the Invention

The compound according to the present invention may be easily synthesized and exhibit effects of inhibiting melanin generation and pigmentation with no adverse effect on the skin, therefore, a composition including the above compound can be used as a skin whitening composition.

### PREFERRED EMBODIMENTS FOR CARRYING OUT THE INVENTION

Hereinafter, preferable embodiments of the present invention will be described in detail with reference to the following examples and experimental examples.

However, these examples and experimental examples are proposed for illustrative purposes only and the scope of the present invention is not particularly limited thereto.

### Example 1

### Preparation of 1,3-bis-butyryloxy-benzene

In a round bottom flask having two openings, 10 mmol of 1,3-benzenediol was poured at room temperature. Then, 50 ml of dichloromethane was introduced into the flask, and cooled using an ice bath. As a catalyst, dimethylaminopyridine (2 mmol) and triethylamine (23 mmol) were introduced into the flask, and butyryl chloride (22 mmol) was slowly added dropwise thereto. After finishing dropping, the mixture was reacted at room temperature for 2 hours. 100 ml of diluted hydrochloric acid solution was added to the reactant, and extraction was executed using 200 ml of dichloromethane. After drying the dichloromethane layer under reduced pressure, separation and purification were executed using silica gel column chromatography (ethyl acetate:hexane=1:20) to produce 1,3-bis-butyryloxy-benzene. The obtained product was subjected to identification by fast atom bombardment mass spectroscopy (FAB-MS).

FAB mass: 251 [M+H]⁺

### Example 2

### Preparation of 1,3-bis-pentanoyloxy-benzene

1,3-bis-pentanyloxy-benzene having the following physical property was synthesized and obtained according to the same procedures as described in Example 1, except that butyryl chloride was replaced by pentanoyl chloride.

FAB mass: 279 [M+H]⁺

### Example 3

### Preparation of 1,3-bis-(3-methyl-2-butenoyloxy)-benzene

1,3-bis-(3-methyl-2-butenoyloxy)-benzene having the following physical property was synthesized and obtained according to the same procedures as described in Example 1, except that butyryl chloride was replaced by 3-methyl-2-butenoyl chloride.

FAB mass: 275 [M+H]⁺

### Example 4

### Preparation of 1,3-bis-octanoyloxy-benzene

1,3-bis-octanoyloxy-benzene having the following physical property was synthesized and obtained according to the same procedures as described in Example 1, except that butyryl chloride was replaced by octanoyl chloride.

FAB mass: 363 [M+H]⁺

### Example 5

### Preparation of 1,3-bis-(2-ethyl-hexanoyloxy)-benzene

1,3-bis-(2-ethyl-hexanoyloxy)-benzene having the following physical property was synthesized and obtained according to the same procedures as described in Example 1, except that butyryl chloride was replaced by 2-ethyl-hexanoyl chloride.

FAB mass: 363 [M+H]⁺

### Example 6

### Preparation of 1,3-bis-decanoyloxy-benzene

1,3-bis-decanoyloxy-benzene having the following physical property was synthesized and obtained according to the same procedures as described in Example 1, except that butyryl chloride was replaced by decanoyl chloride. Characteristics of the product are shown below.

FAB mass: 419 [M+H]⁺

### Example 7

### Preparation of 1,3-bis-lauroyloxy-benzene

1,3-bis-lauroyloxy-benzene having the following physical property was synthesized and obtained according to the same procedures as described in Example 1, except that butyryl chloride was replaced by lauroyl chloride.

FAB mass: 475 [M+H]⁺

### Example 8

### Preparation of 1,3-bis-(2-ethylhexanoyloxy)-naphthalene

1,3-bis-(2-ethylhexanoyloxy)-naphthalene having the following physical property was synthesized and obtained according to the same procedures as described in Example 5, except that 1,3-benzenediol was replaced by 1,3-naphthalenediol.

FAB mass: 413 [M+H]⁺

### Example 9

### Preparation of 2,4-bis-(2-ethylhexanoyloxy)-pyridine

2,4-bis-(2-ethylhexanoyloxy)-pyridine having the following physical property was synthesized and obtained according to the same procedures as described in Example 5, except that 1,3-benzenediol was replaced by 2,4-pyridinediol.

FAB mass: 362 [M+H]⁺

### Example 10

### Preparation of 1,2,3-tris-(2-ethylhexanoyloxy)-benzene

In a round bottom flask having two openings, 10 mmol of 1,2,3-benzenetriol was poured at room temperature. Then, 50 ml of dichloromethane was introduced into the flask, and cooled using an ice bath. As a catalyst, dimethylaminopyridine (3 mmol) and triethylamine (33 mmol) were introduced into the flask, and 2-ethylhexanoyl chloride (33 mmol) was slowly added dropwise thereto. After finishing dropping, the mixture was reacted at room temperature for 2 hours. 100 ml of diluted hydrochloric acid solution was added to the reactant, and extraction was executed using 200 ml of dichloromethane. After drying the dichloromethane layer under reduced pressure, separation and purification were executed using silica gel column chromatography (ethyl acetate:hexane=1:40) to produce 1,2,3-tris-(2-ethylhexanoyloxy)-benzene. The obtained product was subjected to identification by fast atom bombardment mass spectroscopy (hereinafter, referred to as ‘FAB-MS’).

FAB mass: 505 [M+H]⁺

### Example 11

### Preparation of 3,4′,5-tris-(2-ethylhexanoyloxy)-stilbene

3,4′,5-tris-(2-ethylhexanoyloxy)-stilbene having the following physical property was synthesized and obtained according to the same procedures as described in Example 10, except that 1,2,3-benzenetriol was replaced by resveratrol.

FAB mass: 607 [M+H]⁺

### Example 12

### Preparation of 4′,5,7-tris-(2-ethylhexanoyloxy)-isoflavone

4′,5,7-tris-(2-ethylhexanoyloxy)-isoflavone having the following physical property was synthesized and obtained according to the same procedures as described in Example 10, except that 1,2,3-benzenetriol was replaced by genistein.

FAB mass: 649 [M+H]⁺

### Example 13

### Preparation of 3,5,7-tris-(2-ethylhexanoyloxy)-flavone

3,5,7-tris-(2-ethylhexanoyloxy)-flavone having the following physical property was synthesized and obtained according to the same procedures as described in Example 10, except that 1,2,3-benzenetriol was replaced by galangin.

FAB mass: 649 [M+H]⁺

### Example 14

### Preparation of 5,7,4′-tris-(2-ethylhexanoyloxy)-flavone

5,7,4′-tris-(2-ethylhexanoyloxy)-flavone having the following physical property was synthesized and obtained according to the same procedures as described in Example 10, except that 1,2,3-benzenetriol was replaced by apigenin.

FAB mass: 649 [M+H]⁺

### Example 15

### Preparation of 5,6,7-tris-(2-ethylhexanoyloxy)-flavone

5,7,4′-tris-(2-ethylhexanoyloxy)-flavone having the following physical property was synthesized and obtained according to the same procedures as described in Example 10, except that 1,2,3-benzenetriol was replaced by baicalein.

FAB mass: 649 [M+H]⁺

### Example 16

### Preparation of 4′,5,7-tris-(2-ethylhexanoyloxy)-flavanone

4′,5,7-tris-(2-ethylhexanoyloxy)-flavanone having the following physical property was synthesized and obtained according to the same procedures as described in Example 10, except that 1,2,3-benzenetriol was replaced by 4,5,7-trihydroxyflavanone.

FAB mass: 651 [M+H]⁺

### Example 17

### Preparation of 3,5,7,4′-tetrakis-(2-ethylhexanoyloxy)-flavone

In a round bottom flask having two openings, 10 mmol of kaempferol was poured at room temperature. Then, 50 ml of dichloromethane was introduced into the flask, and cooled using an ice bath. As a catalyst, dimethylaminopyridine (4 mmol) and triethylamine (44 mmol) were introduced into the flask, and 2-ethylhexanoyl chloride (44 mmol) was slowly added dropwise thereto. After finishing dropping, the mixture was reacted at room temperature for 2 hours. 100 ml of diluted hydrochloric acid solution was added to the reactant, and extraction was executed using 200 ml of dichloromethane. After drying the dichloromethane layer under reduced pressure, separation and purification were executed using silica gel column chromatography (ethyl acetate:hexane=1:50) to produce 3.5.7.4′-tetrakis-(2-ethylhexanoyloxy)-flavone. The obtained product was subjected to identification by fast atom bombardment mass spectroscopy (FAB-MS).

FAB mass: 791 [M+H]⁺

### Example 18

### Preparation of 3,4,3′,5′-tetrakis-(2-ethylhexanoyloxy)-stilbene

3,4,3′,5′-tetrakis-(2-ethylhexanoyloxy)-stilbene having the following physical property was synthesized and obtained according to the same procedures as described in Example 17, except that kaempferol was replaced by piceatannol.

FAB mass: 749 [M+H]⁺

### Example 19

### Preparation of 3,3′,4′,5,7-pentakis-(2-ethylhexanoyloxy)-flavane

In a round bottom flask having two openings, 10 mmol of catechin was poured at room temperature. Then, 50 ml of dichloromethane was introduced into the flask, and cooled using an ice bath. As a catalyst, dimethylaminopyridine (5 mmol) and triethylamine (55 mmol) were introduced into the flask, and 2-ethylhexanoyl chloride (55 mmol) was slowly added dropwise thereto. After finishing dropping, the mixture was reacted at room temperature for 2 hours. 100 ml of diluted hydrochloric acid solution was added to the reactant, and extraction was executed using 200 ml of dichloromethane. After drying the dichloromethane layer under reduced pressure, separation and purification were executed using silica gel column chromatography (ethyl acetate:hexane=1:60) to produce 3,3′,4′,5,7-pentakis-(2-ethylhexanoyloxy)-flavane. The obtained product was subjected to identification by fast atom bombardment mass spectroscopy (hereinafter, referred to as ‘FAB-MS’).

FAB mass: 921 [M+H]⁺

### Experimental Example 1

### Measurement of Melanin Generation Inhibitory Effects

Each of the compounds prepared according to the procedures described in Examples 1 to 19 and hydroquinone were added to a culture solution containing B-16 mouse melanoma cells, followed by experiments to determine whitening effects in terms of cells (Lotan R., Lotan D. Cancer Res., 40, pp. 3345-3350, 1980). After treating the compounds to have final concentrations of 5 μl/ml and 20 μl/ml, respectively, each of these compounds was added to a culture medium containing B-16 melanoma cells and cultured for 3 days. Then, these cells were treated using trypsin, separated from a culture vessel and centrifuged, then, melanin was extracted from the cells. Melanin was dissolved by adding 1 ml of 1N sodium hydroxide solution to the extract and subjected to measurement of absorbance at 475 nm, and an amount of the generated melanin was expressed by an absorbance per unit number of cells (10⁶ cells) (this procedure was repeated three times). An amount of the generated melanin relative to control was calculated as an inhibition rate (%), and results thereof are shown in Table 1 below.

TABLE 1
Melanin generation inhibitory effects in terms of cells

| | Concentration | Inhibition rate |
|---|---|---|
| Sample | (μg/ml) | (%) |
| Control | — | — |
| Hydroquinone | 1 | 52 |
| | 5 | Apoptosis |
| Material in Example 1 | 5 | 43 |
| | 20 | 68 |
| Material in Example 2 | 5 | 52 |
| | 20 | 85 |
| Material in Example 3 | 5 | 53 |
| | 20 | 83 |
| Material in Example 4 | 5 | 41 |
| | 20 | 71 |
| Material in Example 5 | 5 | 45 |
| | 20 | 79 |
| Material in Example 6 | 5 | 32 |
| | 20 | 53 |
| Material in Example 7 | 5 | 21 |
| | 20 | 43 |
| Material in Example 8 | 5 | 35 |
| | 20 | 63 |
| Material in Example 9 | 5 | 41 |
| | 20 | 67 |
| Material in Example 10 | 5 | 38 |
| | 20 | 65 |
| Material in Example 11 | 5 | 31 |
| | 20 | 54 |
| Material in Example 12 | 5 | 35 |
| | 20 | 65 |
| Material in Example 13 | 5 | 30 |
| | 20 | 63 |
| Material in Example 14 | 5 | 31 |
| | 20 | 57 |
| Material in Example 15 | 5 | 39 |
| | 20 | 72 |
| Material in Example 16 | 5 | 41 |
| | 20 | 59 |
| Material in Example 17 | 5 | 32 |
| | 20 | 69 |
| Material in Example 18 | 5 | 47 |
| | 20 | 72 |
| Material in Example 19 | 5 | 39 |
| | 20 | 69 |

With regard to cultured mousemelanoma cells, all of the compounds exhibited melanin generation inhibitory effects substantially equal to those of hydroquinone. Although hydroquinone has strong effects of inhibiting melanin generation at a low concentration, it cannot undergo experiments at 1 μg/ml or more due to serious cytotoxicity. On the other hand, the above compounds do not exhibit cytotoxicity even at a concentration of 20 μg/ml to thus have more excellent melanin generation inhibitory effects than the hydroquinone.

### Preparative Example 1

### Preparation of Emollient Toner

Formulation 1 and comparative formulation 1 having the following constitutional compositions listed in Table 2 below were prepared.

TABLE 2

| Composition | Formulation 1 | | | | Comparative |
|---|---|---|---|---|---|
| (wt. %) | a | b | c | d | Formulation 1 |
| Material in | 0.1 | — | — | — | — |
| Example 5 | | | | | |
| Material in | — | 0.1 | — | — | — |
| Example 11 | | | | | |
| Material in | — | — | 0.1 | — | — |
| Example 15 | | | | | |
| Material in | — | — | — | 0.1 | — |
| Example 17 | | | | | |
| Ethanol | 10.0 | 10.0 | 10.0 | 10.0 | 10.0 |
| Polyoxyethylene | 1.0 | 1.0 | 1.0 | 1.0 | 1.0 |
| hardened castor | | | | | |
| oil | | | | | |
| Methyl | 0.2 | 0.2 | 0.2 | 0.2 | 0.2 |
| paraoxybenzoate | | | | | |
| Glycerin | 5.0 | 5.0 | 5.0 | 5.0 | 5.0 |
| 1,3-butylene- | 6.0 | 6.0 | 6.0 | 6.0 | 6.0 |
| glycol | | | | | |
| Flavor | Proper | Proper | Proper | Proper | Proper |
| | quantity | quantity | quantity | quantity | quantity |
| Purified water | to 100 | to 100 | to 100 | to 100 | to 100 |

### Preparative Example 2

### Preparation of Nourishing Toner

Formulation 2 and comparative formulation 2 having the following constitutional compositions listed in Table 3 below were prepared

TABLE 3

| Composition | Formulation 2 | | | | Comparative |
|---|---|---|---|---|---|
| (wt. %) | a | b | c | d | Formulation 2 |
| Material in | 0.1 | — | — | — | — |
| Example 5 | | | | | |
| Material in | — | 0.1 | — | — | — |
| Example 11 | | | | | |
| Material in | — | — | 0.1 | — | — |
| Example 15 | | | | | |
| Material in | — | — | — | 0.1 | — |
| Example 17 | | | | | |
| Polyoxyethylene | 1.0 | 1.0 | 1.0 | 1.0 | 1.0 |
| hardened castor | | | | | |
| oil | | | | | |
| Methyl | 0.2 | 0.2 | 0.2 | 0.2 | 0.2 |
| paraoxybenzoate | | | | | |
| Glycerin | 6.0 | 6.0 | 6.0 | 6.0 | 6.0 |
| 1,3-butylene- | 5.0 | 5.0 | 5.0 | 5.0 | 5.0 |
| glycol | | | | | |
| Carbomer | 0.2 | 0.2 | 0.2 | 0.2 | 0.2 |
| Triethanolamine | 0.3 | 0.3 | 0.3 | 0.3 | 0.3 |
| Propyleneglycol | 5.0 | 5.0 | 5.0 | 5.0 | 5.0 |
| Ethanol | 3.2 | 3.2 | 3.2 | 3.2 | 3.2 |
| Carboxyvinyl | 0.1 | 0.1 | 0.1 | 0.1 | 0.1 |
| polymer | | | | | |
| Flavor | Proper | Proper | Proper | Proper | Proper |
| | quantity | quantity | quantity | quantity | quantity |
| Purified water | to 100 | to 100 | to 100 | to 100 | to 100 |

### Preparative Example 3

### Preparation of Cream

Formulation 3 and comparative formulation 2 having the following constitutional compositions listed in Table 4 below were prepared.

TABLE 4

| Composition | Formulation 3 | | | | Comparative |
|---|---|---|---|---|---|
| (wt. %) | a | b | c | d | Formulation 3 |
| Material in | 0.1 | — | — | — | — |
| Example 5 | | | | | |
| Material in | — | 0.1 | — | — | — |
| Example 11 | | | | | |
| Material in | — | — | 0.1 | — | — |
| Example 15 | | | | | |
| Material in | — | — | — | 0.1 | — |
| Example 17 | | | | | |
| Cetanol | 2.0 | 2.0 | 2.0 | 2.0 | 2.0 |
| PEG-20 | 1.0 | 1.0 | 1.0 | 1.0 | 1.0 |
| Sorbitan | 1.0 | 1.0 | 1.0 | 1.0 | 1.0 |
| monostearate | | | | | |
| Mineral oil | 10.0 | 10.0 | 10.0 | 10.0 | 10.0 |
| Trioctanoate | 5.0 | 5.0 | 5.0 | 5.0 | 5.0 |
| Triethanolamine | 0.5 | 0.5 | 0.5 | 0.5 | 0.5 |
| Carbomer | 0.2 | 0.2 | 0.2 | 0.2 | 0.2 |
| Glycerin | 5.0 | 5.0 | 5.0 | 5.0 | 5.0 |
| Propyleneglycol | 3.0 | 3.0 | 3.0 | 3.0 | 3.0 |
| Preservative | Proper | Proper | Proper | Proper | Proper |
| | quantity | quantity | quantity | quantity | quantity |
| Flavor | Proper | Proper | Proper | Proper | Proper |
| | quantity | quantity | quantity | quantity | quantity |
| Purified water | to 100 | to 100 | to 100 | to 100 | to 100 |

### Preparative Example 4

### Preparation of Topical Skin Ointment

Formulation 4 and comparative formulation 4 having the following constitutional compositions listed in Table 5 below were prepared.

TABLE 5

| Composition | Formulation 4 | | | | Comparative |
|---|---|---|---|---|---|
| (wt. %) | a | b | c | d | Formulation 4 |
| Material in | 0.1 | — | — | — | — |
| Example 5 | | | | | |
| Material in | — | 0.1 | — | — | — |
| Example 11 | | | | | |
| Material in | — | — | 0.1 | — | — |
| Example 15 | | | | | |
| Material in | — | — | — | 0.1 | — |
| Example 17 | | | | | |
| Diethyl sebacate | 8.0 | 8.0 | 8.0 | 8.0 | 8.0 |
| Hard lead | 5.0 | 5.0 | 5.0 | 5.0 | 5.0 |
| Polyoxyethylene | 6.0 | 6.0 | 6.0 | 6.0 | 6.0 |
| oleylether | | | | | |
| phosphate | | | | | |
| Sodium benzoate | Proper | Proper | Proper | Proper | Proper |
| | quantity | quantity | quantity | quantity | quantity |
| Vaseline | to 100 | to 100 | to 100 | to 100 | to 100 |

### Preparative Example 5

### Preparation of Essence

Formulation 5 and comparative formulation 5 having the following constitutional compositions listed in Table 6 below were prepared.

TABLE 6

| Composition | Formulation 5 | | | | Comparative |
|---|---|---|---|---|---|
| (wt. %) | a | b | c | d | Formulation 5 |
| Material in | 0.5 | — | — | — | — |
| Example 5 | | | | | |
| Material in | — | 0.5 | — | — | — |
| Example 11 | | | | | |
| Material in | — | — | 0.5 | — | — |
| Example 15 | | | | | |
| Material in | — | — | — | 0.5 | — |
| Example 17 | | | | | |
| Propyleneglycol | 10.0 | 10.0 | 10.0 | 10.0 | 10.0 |
| Glycerin | 10.0 | 10.0 | 10.0 | 10.0 | 10.0 |
| Sodium | 5.0 | 5.0 | 5.0 | 5.0 | 5.0 |
| hyaluronate | | | | | |
| solution (1%) | | | | | |
| Ethanol | 5.0 | 5.0 | 5.0 | 5.0 | 5.0 |
| Polyoxyethylene | 1.0 | 1.0 | 1.0 | 1.0 | 1.0 |
| hardened castor | | | | | |
| oil | | | | | |
| Methyl | 0.1 | 0.1 | 0.1 | 0.1 | 0.1 |
| paraoxybenzoate | | | | | |
| Carbomer | 0.4 | 0.4 | 0.4 | 0.4 | 0.4 |
| Flavor | Proper | Proper | Proper | Proper | Proper |
| | quantity | quantity | quantity | quantity | quantity |
| Purified water | to 100 | to 100 | to 100 | to 100 | to 100 |

### Preparative Example 6

### Preparation of Pack

Formulation 6 and comparative formulation 6 having the following constitutional compositions listed in Table 7 below were prepared.

TABLE 7

| Composition | Formulation 6 | | | | Comparative |
|---|---|---|---|---|---|
| (wt. %) | a | b | c | d | Formulation 6 |
| Material in | 0.1 | — | — | — | — |
| Example 5 | | | | | |
| Material in | — | 0.1 | — | — | — |
| Example 11 | | | | | |
| Material in | — | — | 0.1 | — | — |
| Example 15 | | | | | |
| Material in | — | — | — | 0.1 | — |
| Example 17 | | | | | |
| Glycerin | 5.0 | 5.0 | 5.0 | 5.0 | 5.0 |
| Propyleneglycol | 4.0 | 4.0 | 4.0 | 4.0 | 4.0 |
| Polyvinylalcohol | 15.0 | 15.0 | 15.0 | 15.0 | 15.0 |
| Ethanol | 8.0 | 8.0 | 8.0 | 8.0 | 8.0 |
| Polyoxyethylene | 1.0 | 1.0 | 1.0 | 1.0 | 1.0 |
| hardened castor | | | | | |
| oil | | | | | |
| Polyoxyethylene | 1.0 | 1.0 | 1.0 | 1.0 | 1.0 |
| oleylether | | | | | |
| phosphate | | | | | |
| Methyl | 0.2 | 0.2 | 0.2 | 0.2 | 0.2 |
| paraoxybenzoate | | | | | |
| Flavor | Proper | Proper | Proper | Proper | Proper |
| | quantity | quantity | quantity | quantity | quantity |
| Purified water | to 100 | to 100 | to 100 | to 100 | to 100 |

### Experimental Example 2

### Determination of Pigmentation Inhibitory Effect

In order to verify the pigmentation inhibitory effects of formulations 1 to 6 as well as comparative formulations 1 to 6 prepared in Preparative Examples 1 to 6, respectively, experiments were executed according to the following procedures.

First, 80 healthy panels were selected. An aluminum foil having two lanes of holes, wherein each hole has a diameter of 7 mm and each lane includes seven (7) holes formed therein, was attached to both forearms of every panel. Then, using an artificial solar radiation device (ORIEL solar simulator 1000 W), light with a radiation intensity of about 60 mJ/cm² was emitted at a distance of 10 cm apart from the arm. Before light emission, an irradiating portion was sufficiently washed. From 3 days before the light emission up to 8 weeks after the light emission, formulations 1 to 6 and comparative formulations 1 to 6 were applied in pairs to the same lane of holes twice per day. Pack products of the formulation 6 and comparative formulation 6 were removed when passing 15 minutes after application.

The panels were divided into four (4) groups each of which having 20 persons, and subjected to experiments. Whitening effects were determined by visibly monitoring a degree of pigmentation of the formulations and comparative formulations in each of the panels. Compared to the comparative formulations, extents of pigmentation inhibition of the inventive formulations ware evaluated in three levels including noticeable efficiency, efficiency, and inefficiency, and results of such evaluation are shown in Table 8 below.

TABLE 8
Pigmentation inhibitory effects

| | | Noticeable | | |
|---|---|---|---|---|
| | | efficiency | Efficiency | Inefficiency |
| Test material | | (persons) | (persons) | (persons) |
| Comparative | | 0 | 2 | 18 |
| Formulation 1 | | | | |
| Formulation 1 | a | 4 | 7 | 9 |
| | b | 5 | 9 | 6 |
| | c | 5 | 8 | 7 |
| | d | 4 | 8 | 8 |
| Comparative | | 0 | 4 | 16 |
| Formulation 2 | | | | |
| Formulation 2 | a | 5 | 9 | 6 |
| | b | 4 | 9 | 7 |
| | c | 6 | 9 | 5 |
| | d | 5 | 10 | 5 |
| Comparative | | 0 | 4 | 16 |
| Formulation 3 | | | | |
| Formulation 3 | a | 5 | 11 | 4 |
| | b | 9 | 5 | 6 |
| | c | 6 | 9 | 5 |
| | d | 5 | 7 | 8 |
| Comparative | | 0 | 2 | 18 |
| Formulation 4 | | | | |
| Formulation 4 | a | 5 | 9 | 6 |
| | b | 5 | 9 | 6 |
| | c | 4 | 10 | 6 |
| | d | 6 | 9 | 5 |
| Comparative | | 0 | 3 | 17 |
| Formulation 5 | | | | |
| Formulation 5 | a | 5 | 7 | 8 |
| | b | 6 | 6 | 8 |
| | c | 4 | 11 | 5 |
| | d | 5 | 11 | 4 |
| Comparative | | 0 | 3 | 17 |
| Formulation 6 | | | | |
| Formulation 6 | a | 8 | 6 | 6 |
| | b | 6 | 7 | 7 |
| | c | 7 | 9 | 4 |
| | d | 5 | 9 | 6 |

As shown in the above Table 8, cosmetics prepared of formulations 1 to 6 containing the present inventive materials exhibited remarkably excellent skin whitening effects, compared to conventional cosmetics.

### MODE FOR CARRYING OUT THE INVENTION

The present invention provides a cyclic derivative compound having a chemical structure represented by Formula I below or a pharmacologically acceptable salt thereof, with excellent whitening effects:

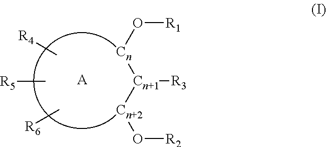

wherein

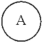

is derived from an aromatic cyclic compound,

Cₙ, Cₙ₊₁ and Cₙ₊₂ are three neighboring carbon atoms present in the aromatic cyclic compound, wherein n is a positive integer,

R₁ and R₂ are each a saturated or unsaturated straight or branched alkyl or acyl group,

R₃, R₄, R₅ and R₆ are each independently at least one substituent selected from a group consisting of hydrogen, alkyl, alkoxy, acyloxy, acyloxymethyl, oxo, hydroxy, vinyl, nitrile, carboxaldehyde, carbonitrile and aldehyde.

Herein, the cyclic compound is any aromatic compound, may have a structure of coupling 1 to 3 or more rings, and may be a homo- or hetero-cyclic compound. Preferably, the heterocyclic compound may contain oxygen or nitrogen in the ring structure, or may contain both oxygen and nitrogen in the ring structure.

The compound represented by Formula I is not particularly limited but may include, for example, benzene, pyridine, pyridazine, pyrimidine, indene, indane, benzofuran, benzothiopen, indole, benzimidazole, benzothiazole, purine, quinoline, isoquinoline, kumarine, cineoline, quinoxaline, flavone, flavane, bibenzyl, 1,3-diphenyl-propane, 1,3-diphenyl-2-propene, or the like.

Further, in the above Formula I,

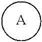

may be derived from a cyclic compound having at least two hydroxyl groups coupled thereto, respectively, and preferably, derived from a cyclic compound that includes one or two 5-membered or 6-membered ring (s) coupled thereto and has hydroxyl groups coupled to at least two or more, preferably, 2 to 6 neighboring carbon atoms in the structure thereof.

In the above Formula I, R₁ and R₂ are each a saturated or unsaturated straight or branched alkyl or acyl group, preferably, C₅ to C₁₀ saturated or unsaturated straight or branched alkyl or acyl group. In particular, if both R₁ and R₂ are alkyl groups, these may be C₄ to C₁₀, and more preferably, C₄ to C₈ saturated or unsaturated straight or branched alkyl groups. Alternatively, if both R₁ and R₂ are acyl groups, these may be C₃ to C₁₂, and more preferably, C₄ to C₁₀ saturated or unsaturated straight or branched acyl group.

R₃, R₄, R₅ and R₆ are the same or different from each other, and may be selected from a group consisting of hydrogen, hydroxy, vinyl, oxo, and alkyl, alkoxy, acyloxy, acyloxymethyl, nitrile, carboxaldehyde, carbonitrile and aldehyde having 1 to 30 carbon atoms, however, it is not necessarily limited thereto.

Cₙ, Cₙ₊₁ and Cₙ₊₂ are respectively three adjacent carbon atoms present in the above cyclic compound, wherein n is a positive number and may be determined within a total number of atoms included in a ring. For instance, if

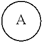

is derived from benzene, n may have the largest number of 4.

Hereinafter, the present invention will be described in more details.

As shown in the following reaction scheme, the inventive compound is an aromatic cyclic compound, preferably, a homo or heterocyclic compound including one or two 5-membered or 6-membered ring(s) coupled thereto, and may be prepared by dissolving a compound with a structure in which hydroxyl groups are bond to at least two or more carbon atoms in a proper solvent, mixing it with at least one or two or more different acyl halides or alkyl halides, and slowly dropping it with desired equivalence ratio to occur esterification or etherification with the hydroxyl groups in the circular structure, or otherwise, further executing hydrogenation of the reaction product formed in the above reaction. In this regard, the organic solvent useable herein is not particularly limited but may include, for example, dichloromethane, chloroform, tetrahydrofuran, acetonitrile, dimethyl formamide, pyridine, or the like. In particular, in the case of etherification, sodium hydride (NaH) or potassium carbonate (K₂CO₃) may be used as a reaction catalyst.

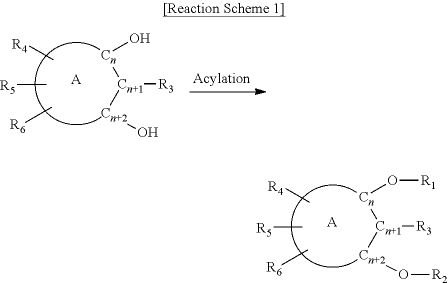

In the above Reaction Scheme 1, R₁ and R₂ are C₃₋₁₂ saturated or unsaturated acyl groups and may be either a straight type or a branched type group.

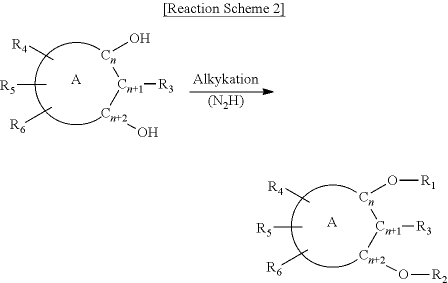

In the above Reaction Scheme 2, R₁ and R₂ are C₃₋₁₀ saturated or unsaturated alkyl groups and may be either a straight type or a branched type group.

Particular examples of the compound represented by Formula I according to the present invention may include, among materials prepared according to Reaction Schemes 1 and 2, the following designated materials formed when a cyclic compound, that is,

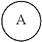

is derived from resorcinol, and R₃, R₄, R₅ and R₆ are hydrogen atoms.

For instance, 1,3-dibutoxybenzene, 1,3-diisobutoxybenzene, 1,3-bis-pentyloxy-benzene, 1,3-bis-(3-methyl-butoxy)-benzene, 1-[2-(2-oxo-butoxy)-cyclohexyloxy]-butan-2-on, 1,3-bis(2,3-dimethyl-butoxy)-benzene, 1,3-bis-(2,4-heptadienyloxy)-benzene, 1,3-bis-(2,4-hexadienyloxy)-benzene, 1,3-bis(2-ethyl-butoxy)-benzene, 1,3-bis(2-ethyl-pentyloxy)-benzene, 1,3-bis-(2-ethyl-hexyloxy)-benzene, 1,3-bis-(2-heptenyloxy)-benzene, 1,3-bis-(2-hexenyloxy)-benzene, 1,3-bis-(2-methyl-butoxy)-benzene, 1,3-bis-(2-methyl-pentyloxy)-benzene, 1,3-bis-(2-methyl-2-pentenyloxy)-benzene, 1,3-bis-(2-pentenyloxy)-benzene, 1,3-bis-(3-heptenyloxy)-benzene, 1,3-bis-(3-methyl-pentyloxy)-benzene, 1,3-bis-(3-methyl-2-butenyloxy)-benzene, 1,3-bis-(3-methyl-3-butenyloxy)-benzene, 1,3-bis-(4-heptenyloxy)-benzene, 1,3-bis-(4-hexenyloxy)-benzene, 1,3-bis-(4-pentenyloxy)-benzene, 1,3-bis-(5-heptenyloxy)-benzene, 1,3-bis-(5-hexenyloxy)-benzene, 1,3-bis-(5-methyl-hexyloxy)-benzene, 1,3-bis-(6-heptenyloxy)-benzene, 1,3-bis-hexyloxy-benzene, 1,3-bis-heptyloxy-benzene, 1,3-bis-octyloxy-benzene, 1,3-bis-nonyloxy-benzene, 1,3-bis-decyloxy-benzene, 1,3-bis-(2-ethyl-hexanoyloxy)-benzene, 1,3-bis-(2-hexanoyloxy)-benzene, 1,3-bis-(2-methyl-2-butenoyloxy)-benzene, 1,3-bis-(2-methyl-2-pentenoyloxy)-benzene, 1,3-bis-(2-methyl-3-butenoyloxy)-benzene, 1,3-bis-(2-methyl-4-pentenoyloxy)-benzene, 1,3-bis-(2-methylbutanoyloxy)-benzene, 1,3-bis-(2,2-dimethylpentanoyloxy)-benzene, 1,3-bis-(2,4-hexadienoyloxy)-benzene, 1,3-bis-(2,4-pentadienoyloxy)-benzene, 1,3-bis-(2-methyl-heptanoyloxy)-benzene, 1,3-bis-(2-methyl-hexanoyloxy)-benzene, 1,3-bis-(2-methyl-pentanoyloxy)-benzene, 1,3-bis-(2-pentenoyloxy)-benzene, 1,3-bis-(3-methyl-2-butenoyloxy)-benzene, 1,3-bis-(3,3-dimethylbutanoyloxy)-benzene, 1,3-bis-(3-hexenoyloxy)-benzene, 1,3-bis-(3-methyl-2-butenoyloxy)-benzene, 1,3-bis-(3-methyl-4-pentenoyloxy)-benzene, 1,3-bis-(3-methylbutanoyloxy)-benzene, 1,3-bis-(3-methylpentanoyloxy)-benzene, 1,3-bis-(3-pentenoyloxy)-benzene, 1,3-bis-(4-methyl-2-pentenoyloxy)-benzene, 1,3-bis-(4-methylhexanoyloxy)-benzene, 1,3-bis-(4-methylpentanoyloxy)-benzene, 1,3-bis-(4-oxo-2-pentenoyloxy)-benzene, 1,3-bis-(4-oxopentanoyloxy)-benzene, 1,3-bis-(4-pentenoyloxy)-benzene, 1,3-bis-(5-oxohexanoyloxy)-benzene, 1,3-bis-(6-oxoheptanoyloxy)-benzene, 1,3-bis-butanoyloxy-benzene, 1,3-bis-pentanoyloxy-benzene, 1,3-bis-hexanoyloxy-benzene, 1,3-bis-heptanoyloxy-benzene, 1,3-bis-octanoyloxy-benzene, 1,3-bis-(2-ethyl-hexanoyloxy)-benzene, 1,3-bis-decanoyloxy-benzene and lauric acid 2-lauroyloxy-benzene, or the like may be used, but it is not particularly limited thereto.

Further, among the materials prepared according to Reaction Scheme 1, the designated materials formed when R₄, R₅ and R₆ are hydrogen atoms and R₁ and R₂ are octanoly groups, may be exemplified as follows.

For instance, 1,3-bis-octanoyloxy-benzene, 2,4-bis-octanoyloxy-pyridine, 4,6-bis-octanoyloxy-pyrimidine, 1,3-bis-octanoyloxy-naphthalene, 1,2,3-tris-octanoyloxy-benzene, 1,3-bis-octanoyloxy-naphthalene, 1,2,3,4-tetra-octanoyloxy-naphthalene, or the like may be used, but it is not particularly limited thereto.

Further, the present invention provides a pharmaceutical composition for external application to the skin in preventing and treating over-pigmentation, freckles, liver spots and age spots, as well as skin whitening, which includes the compound represented by Formula I or a pharmacologically acceptable salt thereof as an effective ingredient.

The compounds represented by the above Formula I may be prepared in the form of a pharmaceutically acceptable salt and/or solvate according to conventional methods known in the related art.

Among the salts, an acid additional salt formed using pharmaceutically acceptable free acids may be usefully employed. The acid additional salt may be prepared according to any conventional method, for example, by dissolving the compound in excess of acid solution to form a salt and precipitating the salt in a water-miscible organic solvent, for example, methanol, ethanol, acetone or acetonitrile. After heating the compound and acid or alcohol (i.e., glycol monomethylether) in water in equimolar amounts, the mixture may be dried through evaporation, or the precipitated salt may be suction-filtered.

In this regard, the free acids used herein may include organic acids and inorganic acids. The inorganic acid may include, for example, hydrochloric acid, phosphoric acid, sulfuric acid, nitric acid, tartaric acid, or the like. Also, the organic acids may include, for example, methane sulfonic acid, p-toluene sulfonic acid, acetic acid, trifluoroacetic acid, citiric acid, maleic acid, succinic acid, oxalic acid, benzoic acid, tartaric acid, fumaric acid, manderic acid, propionic acid, citric acid, lactic acid, glycolic acid, gluconic acid, galaturonic acid, glutamic acid, glutaric acid, glucuronic acid, aspartic acid, ascorbic acid, carbonic acid, vanillic acid, hydroiodic acid, or the like.

Alternatively, it is possible to produce pharmaceutically acceptable metal salts using bases. Alkaline metal or alkali-earth metal salts may be produced by, for example, dissolving the compound in excess of alkaline metal hydroxide or alkali-earth metal hydroxide solution, filtering non-soluble compound salts from the mixture, and evaporating and drying the filtrate. In this regard, the metal salt prepared herein is preferably sodium, potassium or calcium salts in an aspect of pharmaceutical application. Further, a silver salt corresponding thereto may be produced by reacting the alkaline metal or alkali-earth metal salt with any suitable silver salt (i.e., silver nitrate).

Pharmaceutically acceptable salts represented by the above Formula I may include salts of an acidic or basic group possibly present in the compound represented by Formula I, unless otherwise indicated. For instance, such pharmaceutically acceptable salts as described above may include sodium, calcium and potassium salts of hydroxyl group. Further, other pharmaceutically acceptable salts of amino group may include, for example, hydrobromide, sulfate, hydrosulfate, phosphate, hydrophosphate, dihydrophosphate, acetate, succinate, citrate, tartrate, lactate, mandelate, methane sulfonate (mesylate) andp-tolune sulfonate (tosylate), which may be prepared by any general preparation method or process known in the related art.

A composition including the compound of the present invention may further include suitable carriers, excipients or diluents according to any conventional method.

The carrier, excipients and/or diluents included in the composition of the present invention may include, for example, lactose, dextrose, sucrose, sorbitol, mannitol, xylitol, erythritol, maltitol, starch, acacia rubber, alginate, gelatin, calcium phosphate, calcium silicate, cellulose, methyl cellulose, microcrystalline cellulose, polyvinyl pyrrolidone, water, methyl hydroxybenzoate, propyl hydroxybenzoate, talc, magnesium stearate and mineral oil.

According to conventional methods, the composition including the compound of the present invention may be manufactured and used in the form of different formulations including, for example, oral formulations such as powder, granulate, tablet, capsule, suspension, emulsion, syrup, aerosol, etc., topical remedy, suppository or sterile injection solution.

More particular, if the composition is formulated, the formulation may be manufactured using general diluents such as fillers, bulking agents, binders, wetting agents, disintegrants or surfactants, or excipients. Solid formulations for oral administration may include, for example, tablets, pills, powder, granulates, capsules, etc. Such a solid formulation may be manufactured by mixing the above extract with at least one excipient, for example, starch, calcium carbonate, sucrose, lactose, gelatin, etc. In addition, other than a simple excipient, lubricants such as magnesium stearate or talc may also be used. Liquid formulations for oral administration may include, for example, suspension, oral liquid, emulsion, syrup, etc. Other than simple diluents, that is, water, liquid paraffin, etc., often used in the related art, a variety of excipients including, for example, wetting agent, sweetener, flavoring agent, preservative, or the like, may also be included. Formulations for non-oral administration may include sterile aqueous solution, non-aqueous solvent, suspending agent, emulsifier, freeze-drying agent and suppository. Such a non-aqueous solvent or suspending agent used herein may include, for example, propyleneglycol, polyethyleneglycol, vegetable oil such as olive oil, injectable ester such as ethyl oleate, or the like. The suppository may be prepared using witepsol, macrogol, tween 61, cacao butter, lauric butter, glycerogelatin, or the like, as a base material.

A composition for preventing and treating over-pigmentation, freckles, liver spots and age spots, as well as skin whitening, which includes the compound represented by Formula I or its pharmacologically acceptable salt, may be prepared and used as a pharmaceutical composition in the form of topical skin treatment agent formulations capable of being applied to the skin, that is, cream, gel, patch, spray, ointment, plaster, lotion, liniment, paste or cataplasma, however, it is not particularly limited thereto.

A preferable dose of administering the compound represented by Formula I or its pharmacologically acceptable salt according to the present invention may depend upon condition and weight of a patient, severity of disease, type of medicine, administration route, period of time or the like, however, it may be suitably selected by those skilled in the art.

However, in order to achieve desired effects, the inventive compound may be administered in a dose of 0.0001 to 10 mg/kg per day, and preferably, 0.001 to 10 mg/kg per day. The compound may be administered once or separately administered in several times per day. Such an administration dose as described above does not limit the scope of the present invention at all aspects thereof.

With regard to the pharmaceutical composition for external application to the skin, mixing amounts of the inventive compounds may range from 0.0001 to 20 wt. %, and preferably, 0.001 to 10 wt. % to a total weight of the composition. If the amount is less than 0.001 wt. %, whitening effects may be deteriorated. When the amount exceeds 20 wt. %, it is difficult to expect to an increase in whitening effects even it the compound is mixed.

Further, a pharmaceutical administration mode of the inventive compound may include using the compound in the form of pharmaceutically acceptable salts thereof, and using the compound alone or as a combination and/or a simple mixture together with other pharmaceutically active compounds.

The present invention may provide a cosmetic composition for prevention and improvement of over-pigmentation, freckles, liver spots and/or age spots, as well as skin whitening, which includes the compound represented by the above Formula I or its pharmacologically available salt according to the present invention as an effective ingredient.

The composition including the compound represented by Formula I or its pharmacologically acceptable salt according to the present invention as an effective ingredient may be employed in a variety of cosmetics and facial cleansers for anti-wrinkle effects, or the like. Products containing the present composition added thereto may include different cosmetics such as cream, lotion, skin, etc., cleansings, facial cleansers, soaps, treatments, cosmetic liquids, or the like.

The cosmetic of the present invention may include a composition selected from a group consisting of water-soluble vitamins, oil-soluble vitamins, polymer peptide, polymer polysaccharides, sphingolipid and seaweed extract.

The water-soluble vitamin may include any one so far as it can be combined with the cosmetic. Preferably, the water-soluble vitamin includes, for example, vitamin B1, vitamin B2, vitamin B6, pyridoxine, pyridoxine hydrochloride, vitamin B12, pantothenic acid, nicotinic acid, nicotinic acid amide, folic acid, vitamin C, vitamin H, or the like. Further, their salts (i.e., thiamine hydrochloride, sodium ascorbate, etc.) or derivatives (i.e., ascorbic acid-2-phosphate sodium salt, ascorbic acid-2-phosphate magnesium salt, etc.) may also be included in the water-soluble vitamins useable in the present invention. The water-soluble vitamin may be obtained by any conventional method including, for example, microbial transformation, purification from culture products of microorganisms, use of enzyme, chemical synthesis, or the like.

The oil-soluble vitamin may include any one so far as it can be combined with the cosmetic. Preferably, vitamin A, carotene, vitamin D2, vitamin D3, vitamin E (dl-alpha tocopherol, d-alpha tocopherol, d-alpha tocopherol), or the like are included. Further, their derivatives (i.e., ascorbyl palmitate, ascorbyl stearate, ascorbyldipalmitate, acetic acid dl-alpha tocopherol, nitotinic acid dl-alpha tocopherol vitamin E, DL-pantothenyl alcohol, D-pantothenyl alcohol, pantothenyl ethylether, etc.) may also be included in the oil-soluble vitamins used in the present invention. The oil-soluble vitamin may be obtained by any conventional method including, for example, microbial transformation, purification from culture products of microorganisms, use of enzyme, chemical synthesis, or the like.

The polymer peptide may include any one so far as it can be combined with the cosmetic. Preferably, collagen, hydrolyzed collagen, gelatin, elastin, hydrolyzed elastin, keratin, or the like are included. The polymer peptide may be obtained by any conventional method including, for example, purification from culture products of microorganisms, use of enzyme, chemical synthesis, or the like. In addition, the polymer peptide may be purified and used from natural substances such as dermis of pig or cow, cultivated silk of silkworm, etc.

The polymer polysaccharides may include any one so far as it can be combined with the cosmetic. Preferably, hydroxyethyl cellulose, xanthan gum, sodium hyaluronate, chondroitin sulfuric acid or its salt (i.e., sodium salt), or the like are included. For instance, chondroitin sulfuric acid or its salt is generally purified and used from mammal or fish.

The sphingolipid may include any one so far as it can be combined with the cosmetic. Preferably, ceramide, phytosphingosine, sphingoglycolipid, or the like are included. The sphingolipid is generally purified from mammal, fish, shellfish, yeast, plants, etc., or otherwise, obtained by chemical synthesis.

The seaweed extract may include any one so far as it can be combined with the cosmetic. Preferably, brown alga extract, red alga extract, green alga extract, or the like are included. Further, carrageenan, alginic acid, sodium alginate and/or potassium alginate purified from the above seaweed extract may also be included in the seaweed extract used in the present invention. The seaweed extract may be purified from seaweed by any conventional method.

The cosmetic of the present invention may optionally include other components typically combined in the cosmetic, in addition to the above essential ingredients.

Such other additional components to be combined with the cosmetic may include, for example, fats and oils, humectants, emollient agent, surfactant, organic and inorganic pigments, organic powder, UV absorber, preservative, fungicide, antioxidant, plant extract, pH adjuster, alcohol, pigment, flavor, blood flow stimulant, cooling agent, adiaphoretic agent, purified water, or the like.

The fats and oils may include, for example, ester oil, hydrocarbon oil, silicon oil, fluorine oil, animal fat, plant oil, or the like.

Particular examples of the ester oil may include glyceryl tri 2-ethylhexanoate, cetyl 2-ethylhexanoate, isopropyl myristate, butyl myristate, isopropyl palmitate, ethyl stearate, octylpalmitate, isocetyl isostearate, butyl stearate, ethyl linoleate, isopropyl linoleate, ethyl oleate, isocetyl myristate, isostearyl myristate, isostearyl palmitate, octyldodecyl myristate, isocetyl isostearate, diethyl cebacate, diisopropyl adipate, isoalkyl neopentanoate, glyceryl tri(capryl, caprylate), trimethylolpropane tri 2-ethyl hexanoate, trimethylolpropanetriisostearate, pentaerythritol tetra 2-ethylhexanoate, cetyl caprylate, decyl laurate, hexyl laurate, decyl myristate, myristyl myristate, cetyl myristate, stearyl stearate, decyl oleate, cetyl ricinoleate, isostearyl laurate, isotridecyl myristate, isocetyl palmitate, octylstearate, isocetylstearate, isodecyloleate, octyldodecyl oleate, octyldodecyl linoleate, isopropyl isostearate, cetostearyl 2-ethylhexanoate, steary 2-ethylhexanoate, hexyl isostearate, ethyleneglycol dioctanoate, ethyleneglycol dioleate, propyleneglycol dicaprlyate, propyleneglycol di(capryl, caprylate), propyleneglycol dicaprylate, neopentylglycol dicaprylate, neopentylglycol dioctanoate, glyceryl tricaprylate, glyceryl triundecylate, glyceryl triisopalmitate, glyceryl triisostearate, octyldodecyl neopentanoate, isostearyl octanoate, octyl isononanoate, hexyldecyl neodecanoate, octyldodecyl neodecanoate, isocetyl isostearate, isostearyl isostearate, octyldecyl isostearate, polyglycerine oleic acid ester, polyglycerine isostearic acid ester, triisocetyl citrate, triisoalkyl citrate, triisooctyl citrate, lauryl lactate, myristyl lactate, cetyl lactate, octyldecyl lactate, triethyl citrate, acetyltriethyl citrate, acetyltributyl citrate, trioctyl citrate, diisostearyl malate, 2-ethylhexyl hydroxystearate, di 2-ethylhexyl succinate, diisobutyl adipate, diisopropyl sebacate, dioctyl sebacate, cholesteryl stearate, cholesteryl isostearate, cholesteryl hydroxystearate, cholesteryl oleate, dihydrocholesteryl oleate, phytosteryl isostearate, phytosteryl oleate, isocetyl 12-stearoylhydroxystearate, stearyl 12-stearoylhydroxystearate, isostearyl 12-stearoylhydroxystearate, or the like.

Particular examples of the hydrocarbon oil may include squalene, liquid paraffin, alpha-olefin oligomer, isoparaffin, ceresin, paraffin, liquid isoparaffin, polybutene, microcrystalline wax, Vaseline, or the like.

Particular examples of the silicon oil may include polymethyl silicone, methylphenyl silicone, methyl cyclopolysiloxane, octamethyl polysiloxane, decamethyl polysiloxane, dodecamethyl cyclosiloxane, dimethylsiloxane-methylcetyloxysiloxane copolymer, dimethylsiloxane-methylstealoxysiloxane copolymer, alkyl modified silicon oil, amino modified silicon oil, or the like.

The fluorine oil may include perfluoropolyether or the like.

Particular examples of the animal fat or plant oil may include avocado oil, almond oil, olive oil, sesame oil, rice bran oil, safflower oil, soy-bean oil, corn oil, rape oil, apricot kernel oil, palm kernel oil, palm oil, castor oil, sunflower oil, grape seed oil, cottonseed oil, palm oil, kukui nut oil, wheat germ oil, rice germ oil, Shea butter, evening primrose oil, macadamia nut oil, meadow foam seed oil, yolk oil, beef tallow, horse oil, mink oil, orange roughy oil, jojoba oil, candela wax, carnauba wax, liquid lanolin, hardened caster oil, or the like.

The humectants may include, for example, water-soluble small molecular polymer humectants, oil-soluble small molecular polymer humectants, water-soluble polymer, oil-soluble polymer, or the like.

The water-soluble small molecular polymer humectants may include serine, glutamine, sorbitol, mannitol, pyrrolidone-sodium carboxylate, glycerin, propyleneglycol, 1,3-butyleneglycol, ethyleneglycol, polyethyleneglycol B (degree of polymerization n=2 or more), polypropyleneglycol (degree of polymerization n=2 or more), polyglycerin B (degree of polymerization n=2 or more), lactic acid, lactate, or the like.

The oil-soluble small molecular polymer humectants may include cholesterol, cholesterol ester, or the like.

The water-soluble polymer may include, for example, carboxyvinyl polymer, polyasparaginate, tragacanth, xanthan gum, methyl cellulose, hydroxymethyl cellulose, hydroxyethyl cellulose, hydroxypropyl cellulose, carboxymethyl cellulose, water-soluble chitin, chitosan, dextrin, or the like.

The oil-soluble polymer may include, for example, polyvinylpyrrolidone-eicosene copolymer, polyvinylpyrrolidone-hexadecene copolymer, nitrocellulose, dextrin fatty acid ester, polymer silicon, or the like.

The emollient agent may include, for example, long chain acyl glutamic acid cholesteryl ester, cholesteryl hydroxystearate, 12-hydroxystearic acid, stearic acid, rosin acid, lanolin fatty acid cholesteryl ester, or the like.

The surfactant may include, for example, non-ionic surfactants, anionic surfactants, cationic surfactants, amphoteric surfactants, or the like.

Particular examples of the non-ionic surfactants may include self-emulsified monostearic acid glycerin, propyleneglycol fatty acid ester, glycerin fatty acid ester, polyglycerin fatty acid ester, sorbitan fatty acid ester, polyoxyethylene (POE) sorbitan fatty acid ester, POE sorbitol fatty acid ester, POE glycerin fatty acid ester, POE alkylether, POE fatty acid ester, POE hardened castor oil, POE castor oil, polyoxyethylene-polyoxypropylene (POE-POP) copolymer, POE-POP alkylether, polyether modified silicon, alkanolamide laurate, alkylamine oxide, hydrogen added soy bean phospholipid, or the like.

The anionic surfactants may include, for example, fatty acid soap, alpha-acylsulfonate, alkyl sulfonate, alkylaryl sulfonate, alkyl naphthalene sulfonate, alkyl sulfate, POE alkylether sulfate, alkylamide sulfate, alkyl phosphate, POE alkyl phosphate, alkylamide phosphate, alkyloylalkyl taurinate, N-acyl amino acid salt, POE alkylether carboxylate, alkylsulfo succinate, sodium alkylsulfoacetate, acylation hydrolyzed collagen peptide salt, perfluoroalkyl phosphoric acid ester, or the like.

The cationic surfactants may include, for example, alkyl trimethyl ammonium chloride, stearyl trimethyl ammonium chloride, stearyl trimethyl ammonium bromide, cetostearyl trimethyl ammonium chloride, distearyl dimethyl ammonium chloride, stearyl dimethylbenzyl ammonium chloride, behenyl trimethyl ammonium bromide, benzalconium chloride, stearic acid diethylaminoethyl amide, stearic acid dimethylaminopropyl amide, lanolin derivative quaternary ammonium salt, or the like.

The amphoteric surfactants may include, for example, carboxybetaine type, amidebetaine type, sulfobetaine type, hydroxysulfobetaine type, amidsulfobetaine type, phosphobetaine type, aminocarboxylate type, imidazoline derivative type or amidamine type amphoteric surfactants, or the like.

The organic or inorganic pigment may include, for example, inorganic pigments such as silicic acid, silicic anhydride, magnesium silicate, talc, sericite, mica, kaolin, Benguela, clay, bentonite, titanium coated mica, bismuth oxychloride, zirconium oxide, magnesium oxide, zinc oxide, titanium oxide, aluminum oxide, calcium sulfate, barium sulfate, magnesium sulfate, calcium carbonate, magnesium carbonate, iron oxide, ultramarine, chromium oxide, chromium hydroxide, calamine and combinations thereof; organic pigments such as polyamide, polyester, polypropylene, polystyrene, polyurethane, vinyl resin, urea resin, phenol resin, fluorine resin, silicon resin, acryl resin, melamine resin, epoxy resin, polycarbonate resin, divinyl benzene-styrene copolymer, silk powder, cellulose, CI pigment yellow, CI pigment orange, etc.; and a combination pigment of the inorganic and organic pigments, or the like.

The organic powder may include, for example, metal soaps such as calcium stearate; alkylphosphoric acid metal salts such as zinc sodium cetylphosphate, zinc laurylphosphate, calcium laurylphosphate, etc.; multi-valent metal salts of acylamino acid such as N-lauroyl-beta-alanine calcium, N-lauroyl-beta-alanine zinc, N-lauroylglycine calcium, etc.; multi-valent metal salts of amide sulfonic acid such as N-lauroyl-taurine calcium, N-palmitoyl-taurine calcium, etc.; N-acyl basic amino acid such as N-epsilon-lauroyl-L-lysine, N-epsilon-palmitoyl lysine, N-alpha-palmitoyl ornithine, N-alpha-lauroyl arginine, N-alpha-hydrogenated beef tallow fatty acid acylarginine, etc.; N-acyl polypeptide such as N-lauroyl glycylglycine, etc.; alpha-amino fatty acid such as alpha-amino caprylic acid, alpha-amino lauric acid, etc.; polyethylene, polypropylene, nylon, polymethyl methacrylate, polystyrene, divinylbenzene-styrene copolymer, ethylene tetrafluoride, or the like.

UV absorber may include, for example, para-aminobenzoic acid, ethyl para-aminobenzoate, amyl para-aminobenzoate, octyl para-aminobenzoate, ethyleneglycol salicylate, phenyl salicylate, octyl salicylate, benzyl salicylate, butylphenyl salicylate, homomentyl salicylate, benzyl cinnamate, para-methoxy cinnamic acid-2-ethoxyethyl, octyl para-methoxy cinnamate, mono-2-ethylhexane glyceryl di-para-methoxy cinnamate, isopropyl para-methoxy cinnamate, diisopropyl-diisopropyl cinnamic acid ester mixture, urocanic acid, ethyl urocanate, hydroxymethoxy benzophenone, hydroxymethoxy benzophenone sulfonic acid and its salt, dihydroxymethoxy benzophenone, sodium dihydroxymethoxy benzophenone disulfonate, dihydroxy benzophenone, tetrahydroxy benzophenone, 4-tert-butyl-4′-methoxydibenzoyl methane, 2,4,6-trianilino-p-(carbo-2-ethylhexyl-1-oxy)-1,3,5-triazine, 2-(2-hydroxy-5-methylphenyl)benzotriazole, or the like.

The fungicide may include, for example, hinoki thiol, trichloric acid, trichlorohydroxydiphenyl ether, chlorohexidine gluconate, phenoxyethanol, resorcine, isopropylmethyl phenol, azulene, salicylic acid, zinc pyrithione, benzalconium chloride, photosensitive element No. 301, mononitro-guaiacol sodium, undecylenic acid, or the like.

The antioxidant may include butylhydroxyanisole, propyl gallate, Eli sorbic acid, or the like.

The pH adjuster may include, for example, citric acid, sodium citrate, malic acid, sodium malate, fumaric acid, sodium fumarate, succinic acid, sodium succinate, sodium hydroxide, sodium hydrogen phosphate, or the like.

The alcohol may include long-chain alcohols such as cetyl alcohol.

Such other additional components to be combined are not particularly limited to those as described above, and any one of these components may be combined within a range that does not damage the purposes and effects of the present invention, preferably, in a range of 0.01 to 5 wt. %, and preferably, 0.01 to 3 wt. % to a total weight of the composition.

The cosmetic of the present invention may be produced in a form of solution, emulsion, viscous mixture, or the like.

The cosmetic composition according to the present invention includes any of the compounds represented by Formula I as effective ingredients, and in addition, any component commonly used in the related art, for example, typical adjuvant and carrier such as a stabilizer, solvate, vitamin, pigment and/or flavor.

The cosmetic composition of the present invention may be formulated in any form of products typically manufactured in the art, for example, emulsion, cream, skin toner, pack, foundation, lotion, cosmetic liquid, hair cosmetics, etc.

More particularly, the cosmetic composition of the present invention may include formulations such as skin lotion, skin softener, skin toner, astringent, lotion, milk lotion, moisture lotion, nourishing lotion, massage cream, nourishing cream, moisture cream, hand cream, foundation, essence, nourishing essence, pack, soap, cleansing foam, cleansing lotion, cleansing cream, body lotion and body cleanser.

When the formulation of the present invention is a paste, cream or gel, animal fiber, plant fiber, wax, paraffin, starch, tragacanth, cellulose derivatives, polyethyleneglycol, silicon, bentonite, silica, talc or zinc oxide may be used as a carrier component.

When the formulation of the present invention is a powder or spray, lactose, talc, silica, aluminum hydroxide, calcium silicate or polyamide powder may be used as a carrier component. In particular, for the spray formulation, a propellant such as chlorofluorohydrocarbon, propane/butane or dimethyl ether may be further included.

When the formulation of the present invention is a solution or emulsion, a solvent, solvate or emulsifier is used as a carrier component. For instance, water, ethanol, isopropanol, ethyl carbonate, ethyl acetate, benzyl alcohol, benzyl benzoate, propyleneglycol, 1,3-butylglycol oil, glycerol aliphatic ester, polyethyleneglycol or sorbitan fatty acid ester may be used.

When the formulation of the present invention is a suspension, a carrier component including, for example: a liquid diluent such as water, ethanol or propyleneglycol; a suspending agent such as ethoxylated isostearyl alcohol, polyoxyethylene sorbitol ester or polyoxyethylene sorbitan ester; microcrystalline cellulose; aluminum meta-hydroxide, bentonite, agar or tragacanth may be used.

When the formulation of the present invention is a surfactant containing cleansing, a carrier composition including, for example, aliphatic alcohol sulfate, aliphatic alcohol ether sulfate, sulfosuccinic acid monoester, acethionate, imidazolinium derivative, methyl taurate, sarcosinate, fatty acid amide ether sulfate, alkyl amidobetaine, aliphatic alcohol, fatty acid glyceride, fatty acid diethanolamide, vegetable oil, linoline derivative or ethoxylated glycerol fatty acid ester, or the like may be used.

1. A derivative or a polyhydroxy cyclic compound represented by Formula I below or a pharmacologically acceptable salt thereof, comprising: 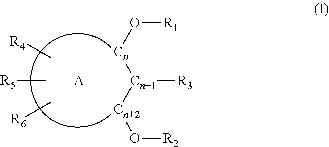
wherein 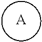
is derived from an aromatic cyclic compound,
Cₙ, Cₙ₊₁ and Cₙ₊₂ are three neighboring carbon atoms present in the aromatic cyclic compound, wherein n is a positive integer,
R₁ and R₂ are each a saturated or unsaturated straight or branched alkyl or acyl group having 3 to 12 carbon atoms, and
R₃, R₄, R₅ and R₆ are each independently at least one substituent selected from a group consisting of hydrogen, oxo, hydroxy, and alkyl, alkoxy, acyloxy, acyloxymethyl, vinyl, nitrile, carboxaldehyde, carbonitrile and aldehyde having 1 to 30 carbon atoms.

2. The derivative or its pharmacologically acceptable salt according to claim 1, wherein the 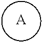
is derived from a cyclic compound having one to three pentagonal or hexagonal ring(s) coupled thereto.

3. The derivative or its pharmacologically acceptable salt according to claim 1, wherein the cyclic compound is a homo- or heterocyclic compound.

4. The derivative or its pharmacologically acceptable salt according to claim 3, wherein the heterocyclic compound contains oxygen, nitrogen, or oxygen and nitrogen in a structure thereof.

5. The derivative or its pharmacologically acceptable salt according to claim 1, wherein the 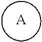
is derived from a cyclic compound selected from a group consisting of benzene, pyridine, naphthalene, flavone, isoflavone, flavane, flavanone, stilbene and coumarin.

6. The derivative or its pharmacologically acceptable salt according to claim 1, wherein the 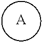
is derived from a cyclic compound having hydroxyl groups coupled to 1 to 3 neighboring carbon atoms.

7. The derivative or its pharmacologically acceptable salt according to claim 1, wherein R₁ and R₂ are each a saturated or unsaturated straight or branched alkyl or acyl group having 3 to 12 carbon atoms.

8. The derivative or its pharmacologically acceptable salt according to claim 1, wherein it is represented by Formula I, and the 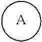
has a ring structure derived from benzene.

9. The derivative or its pharmacologically acceptable salt according to claim 1, wherein it is represented by Formula I, and the 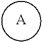
has a ring structure derived from pyridine.

10. The derivative or its pharmacologically acceptable salt according to claim 1, wherein it is represented by Formula I, and the 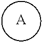
has a ring structure derived from naphthalene.

11. The derivative or its pharmacologically acceptable salt according to claim 1, wherein it is represented by Formula I, and the 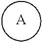
has a ring structure derived from stilbene.

12. The derivative or its pharmacologically acceptable salt according to claim 1, wherein it is represented by Formula I, and the 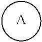
has a ring structure derived from flavone.

13. The derivative or its pharmacologically acceptable salt according to claim 1, wherein it is represented by Formula I, and the 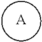
has a ring structure derived from isoflavone.

14. The derivative or its pharmacologically acceptable salt according to claim 1, wherein it is represented by Formula I, and the 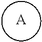
has a ring structure derived from flavane.

15. The derivative or its pharmacologically acceptable salt according to claim 1, wherein it is represented by Formula I, and the 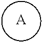
has a ring structure derived from flavanone.

16. A composition for prevention and treatment of over-pigmentation, freckles, liver spots and/or age spots, as well as skin whitening, which includes the compound or its pharmacologically acceptable salt according to any one of claims 1 to 15 as an effective ingredient.

17. The composition according to claim 16, wherein the composition is a formulation selected from cream, gel, patch, spray, ointment, plaster, lotion, liniment, paste or cataplasma, which is a pharmaceutical composition for external application to the skin.

18. A cosmetic composition for prevention and improvement of over-pigmentation, freckles, liver spots and/or age spots, as well as skin whitening, which includes the compound according to any one of claims 1 to 15 as an effective ingredient.

19. The cosmetic composition according to claim 18, wherein the composition is a formulation selected from a group consisting of skin lotion, skin softener, skin toner, astringent, lotion, milk lotion, moisture lotion, nourishing lotion, massage cream, nourishing cream, moisture cream, hand cream, foundation, essence, nourishing essence, pack, soap, cleansing foam, cleansing lotion, cleansing cream, body lotion and body cleanser.
